(19) United States
(12) Patent Application Publication
(10) Pub. No.: US 2015/0265663 A1
(43) Pub. Date: Sep. 24, 2015

(54) METHOD FOR SUPPRESSING ADIPOCYTE FORMATION

(71) Applicant: Maruhachi Muramatsu, Inc., Yaizu-shi (JP)

(72) Inventor: Masayoshi YAMAGUCHI, Shizuoka-shi (JP)

(73) Assignee: Maruhachi Muramatsu, Inc., Yaizu-shi (JP)

(21) Appl. No.: 14/223,336

(22) Filed: Mar. 24, 2014

(51) Int. Cl. A61K 36/02

(52) U.S. Cl. CPC A61K 36/02

(57) ABSTRACT

Provided are a naturally-derived agent for suppressing differentiation into adipocyte, readily available and ingestible in daily dietary life and having little side effects, a method for suppressing differentiation into adipocyte in a subject in need thereof, and the like. The present inventors have confirmed that a water extract of Sargassum horneri has the effect of suppressing differentiation from bone marrow mesenchymal stem cell into adipocyte, thereby accomplishing the present invention.

### TECHNICAL FIELD

The present invention relates to a method for suppressing adipocyte formation in a subject in need thereof, comprising administering a therapeutic effective amount of an agent suppressing differentiation from bone marrow cells into adipocytes to the subject, wherein the agent suppressing differentiation is Sargassum horneri or a processed product thereof, and an agent suppressing differentiation into adipocytes, containing Sargassum horneri or a processed product thereof as an active ingredient.

### BACKGROUND ART

The homeostasis of bone metabolism is regulated by osteoclasts, osteoblasts, and osteocytes (see, for example, Non-patent Documents 1 and 2). Osteoblasts are differentiated and formed from bone marrow mesenchymal stem cells and increase bone formation and calcification. Osteoclasts are differentiated and formed from blood stem cells and contribute to the destruction of bone. Bone tissue is metabolized through physiological mechanisms called the construction and reconstruction thereof by the functions of these cells to maintain the flexibility and elasticity thereof. These processes involve many hormones and cytokines as well as the bone marrow environment and are ingeniously regulated. The failure of these regulatory mechanisms is known to be accompanied by a decrease in the amount of bone to cause a wide range of bone diseases including osteoporosis.

Mesenchymal stem cells in the bone marrow are multipotent stromal cells and known to differentiate into osteoblasts, chondrocytes, cardiomyocytes, adipocytes, and the like (see, for example, Non-patent Documents 3 to 5). The differentiation processes are regulated by complex signal systems, and examples of known factors involved in such signal systems include protein regulators such as bone morphogenic proteins (BMPs), wingless type MMTV integration site proteins, hedgehogs delta/jagged proteins, fibroblastic growth factors, insulin, insulin-like growth factors, and transcription factors for adipocytes and osteoblasts (peroxisome proliferators-activated receptor gamma (PPAγ) and runt-related transcription factor 2 (Runx2)) (see, for example, Non-patent Documents 6 to 9).

As to the differentiation of bone marrow mesenchymal stem cells into adipocytes, it is known that specific hormone-related regulators such as dexamethasone, 3-isobutyl-1-methylxanthine, and insulin are involved in specific differentiation into preadipocytes, but whether mesenchymal stem cells in the bone marrow differentiate into adipocytes or cells other than adipocytes and whether it is possible to suppress differentiation into adipocytes are questions which have increasingly received attention in recent years in connection with a need for the prevention or treatment of obesity in the current aging society.

As ingredients suppressing differentiation into adipocytes, there are proposed a preadipocyte differentiation-suppressing agent containing siphonaxanthin as an active ingredient (see, for example, Patent Document 1), a preadipocyte differentiation-suppressing agent containing one or more extracts of plants selected from the group consisting of Cassia obtusifolia, Platycodon grandiflorum, Phellodendron amurense, Sophora angustifolia, Evodia rutaecarpa, Lonicera japonica, Cassia occidentalis, Glehnia littoralis, and Areca catechu (see, for example, Patent Document 2), a preadipocyte differentiation-suppressing agent containing a cyclic peptide as an active ingredient (see, for example, Patent Document 3), an adipocyte differentiation inhibitor containing a prolactin inhibitor as an active ingredient (see, for example, Patent Document 4), and a peptide having the ability to suppress adipocyte differentiation, consisting of Val-Tyr-Pro and/or Val-Thr-Leu (see, for example, Patent Document 5); however, they cannot be said to have sufficient effects, and there is need for the development of a new ingredient suppressing differentiation into adipocytes.

### PRIOR ART DOCUMENTS

### Patent Documents

### Patent Document 1

Japanese unexamined Patent Application Publication No. 2012-001524

### Patent Document 2

Japanese unexamined Patent Application Publication No. 2005-239659

### Patent Document 3

Japanese unexamined Patent Application Publication No. 2005-220074

### Patent Document 4

Japanese unexamined Patent Application Publication No. 2000-217576

### Patent Document 5

Japanese unexamined Patent Application Publication No. 06-293796

### Non-patent Documents

### Non-Patent Document 1

Raggatt L. J., Partridge N. C. (2010) Cellular and molecular mechanisms of bone remodeling. J. Biol. Chem. 285: 25103-25108.

### Non-Patent Document 2

Chambers T. J., Fuller K. (2011) How are osteoclasts induced to resorb bone? Ann. N. Y. Acad. Sci. 1240: 1-6.

### Non-Patent Document 3

Chen G., Deng C., Li Y. P. (2012) TGF-β and BMP signaling in osteoblast differentiation and bone formation. Int. J. Biol. Sci. 8: 272-288.

### Non-Patent Document 4

Minguel J. J., Erices A., Conget P. (2001) Mesenchymal stem cells. Exp. Biol. Med. 226: 507-520.

### Non-Patent Document 5

Muruganandan S., Roman A. A., Sinal C. J. (2009) Adipocyte differentiation of bone marrow-derived mesenchymal stem cells: cross talk with the osteoblastogenesis program. Cell. Mol. Life Sci. 66: 236-253.

### Non-Patent Document 6

Laudes M. (2011) Role of WNT signalling in the determination of human mesenchymal stem cells into preadipocytes. J. Mol. Endocrinol. 46: R65-72.

### Non-Patent Document 7

Gharibi B., Abraham A. A., Ham J., Evans B. A. (2011) Adenosine receptor subtype expression and activation influence the differentiation of mesenchymal stem cells to osteoblasts and adipocytes. J. Bone Miner. Res. 26: 2112-2124.

### Non-Patent Document 8

Kawai M., Rosen C. J. (2010) PPARy: a circadian transcription factor in adipogenesis and osteogenesis. Nat. Rev. Endocrinol. 6: 629-636.

### Non-Patent Document 9

Wu L., Cai X., Dong H., Jing W., Huang Y., Yang X., Wu Y., Lin Y. (2010) Serum regulates adipogenesis of mesenchymal stem cells via MEK/ERK-dependent PPARy expression and phosphorylation. J. Cell. Mol. Med. 14: 922-932.

### SUMMARY OF THE INVENTION

### Object to be Solved by the Invention

An object of the present invention is to provide a naturally-derived agent suppressing differentiation into adipocytes, readily available and ingestible in daily dietary life and having little side effects, a method for suppressing differentiation into adipocytes in a subject in need thereof, and the like.

### Means to Solve the Object

The present inventors have proceeded with research on various edible marine algae such as Undaria pinnatifida, Eisenia bicyclis, Gelidium amansii, and Ulva pertusa Kjellman, and, they have proposed, as to Sargassum horneri known as a marine alga of the genus Sargassum of the order Fucales of the class Phaeophyceae abundantly produced in shallow sea, a bone quantity-increasing composition exerting an anti-osteoporosis effect, containing a processed product thereof as an active ingredient (Japanese unexamined Patent Application Publication No. 2003-026597), an agent for preventing/ameliorating diabetic pathological conditions, containing Sargassum horneri or a processed product thereof as an active ingredient (Japanese unexamined Patent Application Publication No. 2004-217559), and an NF—KB inhibitor containing Sargassum horneri or a processed product thereof as an active ingredient (Japanese unexamined Patent Application Publication No. 2013-213007). The present inventors have further continued studies and confirmed that a water extract of Sargassum horneri has the effect of suppressing differentiation from bone marrow mesenchymal stem cells into adipocytes, thereby accomplishing the present invention.

Specifically, the present invention relates to the following:

(1) A method for suppressing adipocyte formation in a subject in need thereof, comprising administering a therapeutic effective amount of an agent suppressing differentiation into adipocyte to the subject, wherein the agent suppressing differentiation is Sargassum horneri or a processed product thereof;

(2) The method for suppressing adipocyte formation according to (1) above, wherein the processed product of Sargassum horneri is an extract of Sargassum horneri;

(3) The method for suppressing adipocyte formation according to (2) above, wherein the extract of Sargassum horneri is a water extract of Sargassum horneri;

(4) The method for suppressing adipocyte formation according to (3) above, wherein the water extract of Sargassum horneri is a water extract of Sargassum horneri having a molecular weight of 3,000 or less;

(5) The method for suppressing adipocyte formation according to (1) above, wherein the agent suppressing differentiation into adipocyte is an agent suppressing differentiation from bone marrow cell into preadipocyte;

(6) The method for suppressing adipocyte formation according to (1) above, wherein the agent suppressing differentiation into adipocyte is an agent suppressing differentiation from preadipocyte into adipocyte; and

(7) The method for suppressing adipocyte formation according to (1) above, wherein the subject is one or more selected from mouse, rat, bird, pig, sheep, cattle, cat, dog, primate, and human.

The present invention also relates to the following:

1) An agent suppressing differentiation into adipocyte, comprising Sargassum horneri or a processed product thereof as an active ingredient;

2) The agent suppressing differentiation according to 1) above, wherein the processed product of Sargassum horneri is an extract of Sargassum horneri;

3) The agent suppressing differentiation according to 2) above, wherein the extract of Sargassum horneri is a water extract of Sargassum horneri;

4) The agent suppressing differentiation according to 2) or 3) above, wherein the extract of Sargassum horneri is an extract of Sargassum horneri having a molecular weight of 3,000 or less;

5) An agent suppressing adipocyte formation, comprising the agent suppressing differentiation into adipocytes according to any one of 1) to 4) above;

6) An anti-obesity agent comprising the agent suppressing differentiation into adipocyte according to any one of 1) to 4) above;

7) A functional food or food material for preventing/ameliorating pathological condition due to adipocyte hyperplasia, comprising Sargassum horneri or a processed product thereof as an active ingredient;

8) The functional food or food material for preventing/ameliorating pathological condition due to adipocyte hyperplasia according to 7) above, wherein the processed product of Sargassum horneri is an extract of Sargassum horneri;

9) A feedstuff for preventing/ameliorating pathological condition due to adipocyte hyperplasia, comprising Sargassum horneri or a processed product thereof as an active ingredient;

10) The feedstuff for preventing/ameliorating pathological condition due to adipocyte hyperplasia according to 9) above, wherein the processed product of Sargassum horneri is an extract of Sargassum horneri;

11) Use of Sargassum horneri or a processed product thereof for production of an agent suppressing differentiation into adipocytes;

12) Use of Sargassum horneri or a processed product thereof for production of an agent suppressing adipocyte formation;

13) Sargassum horneri or a processed product thereof for use in an agent suppressing differentiation into adipocytes; and

14) Sargassum horneri or a processed product thereof for use in an agent suppressing adipocyte formation.

### Effect of the Invention

The agent suppressing differentiation into adipocytes according to the present invention is highly safe because it can be obtained by extraction and purification from natural products used for food for years, exerts the fat accumulation-suppressing effect of suppressing fat accumulation in visceral tissue and subcutaneous tissue, the effect of preventing obesity, the effect of suppressing cellulite formation, and the effect of suppressing hyperlipemia, and has an excellent effect enabling oral administration.

### BRIEF DESCRIPTION OF DRAWINGS

FIG. 1 is a graph showing the number of adipocytes formed after performing culture in the differentiation stage into preadipocytes in a culture medium for differentiation into preadipocytes, containing a water extract of Sargassum horneri in a concentration of 0, 5, 10, 25, or 50 μg/mL culture medium and then performing culture in the differentiation stage into adipocytes in a culture medium for differentiation into adipocytes, containing the water extract of Sargassum horneri in a concentration of 0, 5, 10, 25, or 50 μg/mL culture medium.

FIG. 2 is a graph showing the absorbance of cell samples after performing culture in the differentiation stage into preadipocytes in a culture medium for differentiation into preadipocytes, containing a water extract of Sargassum horneri in a concentration of 0, 5, 10, 25, or 50 μg/mL culture medium and then performing culture in the differentiation stage into adipocytes in a culture medium for differentiation into adipocytes, containing the water extract of Sargassum horneri in a concentration of 0, 5, 10, 25, or 50 μg/mL culture medium.

FIG. 3 is a graph showing the absorbance of cells after performing culture in the differentiation stage into preadipocytes in a culture medium for differentiation into preadipocytes, containing a water extract of Sargassum horneri in a concentration of 0, 5, 10, 25, or 50 μg/mL culture medium and then performing culture in the differentiation stage into adipocytes in a culture medium for differentiation into adipocytes, not containing the water extract of Sargassum horneri.

FIG. 4 is a graph showing the absorbance of cells after performing culture in the differentiation stage into preadipocytes in a culture medium for differentiation into preadipocytes, not containing a water extract of Sargassum horneri and then performing culture in the differentiation stage into adipocytes in a culture medium for differentiation into adipocytes, containing the water extract of Sargassum horneri in a concentration of 0, 5, 10, 25, or 50 μg/mL culture medium.

### MODE OF CARRYING OUT THE INVENTION

The method for suppressing adipocyte formation according to the present invention is not particularly limited, provided that it is a method for suppressing adipocyte formation in a subject in need thereof, comprising administering a therapeutic effective amount of an agent suppressing differentiation into adipocytes to the subject, wherein the agent suppressing differentiation is Sargassum horneri or a processed product thereof. Examples of the Sargassum horneri or a processed product thereof can include a water extract of Sargassum horneri obtained by separating a soluble ingredient using water such as room temperature water, hot water, and deionized water, an organic solvent extract of Sargassum horneri obtained by separating a soluble ingredient using an organic solvent such as aqueous alcohol and hexane, a dried product of Sargassum horneri obtained by drying the whole Sargassum horneri, a dried powder of Sargassum horneri obtained by subjecting dried Sargassum horneri to pulverization treatment, and an enzyme-treated product of Sargassum horneri obtained by treating Sargassum horneri using an enzyme such as cellulase; among them, a water extract of Sargassum horneri and an organic solvent extract of Sargassum horneri are preferable, and especially, a water extract of Sargassum horneri can be preferably exemplified.

The agent suppressing differentiation into adipocytes (including mature adipocytes) according to the present invention is not particularly limited, provided that it contains Sargassum horneri or a processed product thereof as an active ingredient. Although the water extract of Sargassum horneri or the organic solvent extract of Sargassum horneri can be used as an active ingredient of the agent suppressing differentiation into adipocytes as they are, these extracts can also be used by further fractionating them into fractions having a high activity of suppressing differentiation into adipocytes by an appropriate purification procedure such as a silica gel column chromatography method, a reverse-phase column chromatography method, a gel filtration chromatography method, and a membrane filter procedure. The fraction having a high activity of suppressing differentiation into adipocytes can be preferably exemplified by that having a molecular weight of 30,000 or less, preferably 10,000 or less, more preferably 5,000 or less, still more preferably 3,000 or less.

Preferred methods for preparing Sargassum horneri or a processed product thereof can include a method which involves preparing an organic solvent extract of Sargassum horneri or a water extract of Sargassum horneri by adding, to raw Sargassum horneri washed with water and then subjected to crushing treatment with a homogenizer or the like, a 1- to 5-fold, preferably 2- to 4-fold, particularly preferably about 3-fold volume of water such as room temperature water, hot water, and deionized water or 5 to 80%, preferably 10 to 40%, more preferably about 20% of any of various organic solvents such as aqueous alcohol (such as methanol, ethanol, and propanol) and hexane for extraction treatment and subjecting the extract to centrifugation treatment at 4,000 to 7,000 g, preferably 5,000 to 6,000 g for 5 to 15 minutes, preferably about 10 minutes to separate a soluble fraction, and a method which involves washing raw Sargassum horneri with water and then drying the resultant by vacuum freeze-drying, solar drying, air drying, hot air drying, heat drying, microwave drying, or the like; however, the method can be preferably exemplified which involves adding, to Sargassum horneri subjected to crushing treatment, an about 3-fold volume of water for extraction treatment and subjecting the extract to centrifugation treatment at 5,000 to 6,000 g for about 10 minutes to separate a soluble fraction. Sargassum horneri for extraction treatment may also be intact Sargassum horneri not subjected to crushing treatment, and in this case, it is preferable to perform crushing treatment with a homogenizer or the like after extraction treatment. When Sargassum horneri is not subjected to processing treatment immediately after collection, it is preferably stored at a temperature as low as 10° C. or below, for example, 4 to 5° C.

A method for confirming whether an agent suppressing differentiation into adipocytes has the effect of suppressing differentiation into adipocytes or not is a method which involves culturing bone marrow mesenchymal stem cells in a culture medium for differentiating bone marrow mesenchymal stem cells into adipocytes, with or without an agent suppressing differentiation to be tested, and then counting the number of formed adipocytes, wherein if the number of cells differentiated into adipocytes with the agent suppressing differentiation to be tested is significantly decreased compared to the number of cells differentiated into adipocytes without the agent suppressing differentiation to be tested, then the agent suppressing differentiation to be tested can be determined to have the effect of suppressing differentiation from bone marrow mesenchymal stem cells into adipocytes (method 1).

Other methods for confirming whether an agent suppressing differentiation into adipocytes has the effect of suppressing differentiation into adipocytes or not include (a) a method which involves culturing bone marrow mesenchymal stem cells in a culture medium for differentiation into preadipocytes, with or without an agent suppressing differentiation to be tested, then culturing the resultant in a culture medium for differentiation into adipocytes, and counting the number of formed adipocytes, wherein if the number of formed adipocytes with the agent suppressing differentiation to be tested in the differentiation stage into preadipocytes is significantly decreased compared to the number of formed adipocytes without the agent suppressing differentiation to be tested in the differentiation stage into preadipocytes, then the agent suppressing differentiation to be tested can be determined to have the effect of suppressing differentiation from bone marrow mesenchymal stem cells into preadipocytes (method 2), or (b) a method which involves culturing bone marrow mesenchymal stem cells in a culture medium for differentiation into preadipocytes, then culturing the preadipocytes in a culture medium, which is the culture medium for differentiation into adipocytes with or without the agent suppressing differentiation to be tested, to cause differentiation into adipocytes, and counting the number of formed adipocytes, wherein if the number of formed adipocytes with the agent suppressing differentiation to be tested in the differentiation stage into adipocytes is significantly decreased compared to the number of formed adipocytes without the agent suppressing differentiation to be tested, then the agent suppressing differentiation to be tested can be determined to have the effect of suppressing differentiation from preadipocytes into adipocytes (method 3).

The culture method for differentiation into preadipocytes can be specifically exemplified by a method which involves culturing bone marrow mesenchymal stem cells in a culture medium for differentiation into preadipocytes, which is a culture medium containing fetal bovine serum, with a cell differentiation-promoting factor such as dexamethasone and 3-isobutyl-1-methylxanthine. The culture method for differentiation into adipocytes can be specifically exemplified by a method which involves culturing the preadipocytes after replacing the medium with a culture medium for differentiation into adipocytes, which is a culture medium containing fetal bovine serum, with a cell differentiation-promoting factor such as insulin.

Examples of the culture period for the differentiation into preadipocytes include 6 to 90 hours, preferably 24 to 72 hours, more preferably 36 to 60 hours, still more preferably 42 to 54 hours, particularly preferably 45 to 51 hours. Examples of the culture period for the differentiation into adipocytes include 1 to 7 days, preferably 2 to 6 days, more preferably 3 to 5 days, still more preferably 3.5 to 5.5 days.

The number of the formed adipocytes can be counted/confirmed by an ordinary method; examples of the method include a method which involves fixing cells wherein the culture medium is removed with a formalin solution, adding Oil Red reagent thereto, and counting the number of stained cells for confirmation, and a method which involves further dissolving the cells stained by adding Oil Red reagent, in isobutyl alcohol to extract an orthochromatic dye taken up in the cells, measuring the resultant extract for absorbance at 490 nm as the absorption wavelength of the Oil Red dye, and counting/confirming the number of formed adipocytes by the amount of the absorbance.

According to the method for suppressing adipocyte formation of the present invention, examples of the subject in need thereof can include a subject in need of suppressing adipocyte formation, a subject in need of treating pathological conditions involving adipocyte hyperplasia, and a subject in need of preventing the development of such pathological conditions. Examples of the pathological conditions involving adipocyte hyperplasia can include fat accumulation, obesity, cellulite formation, and hyperlipemia; particularly, pathological conditions can be mentioned which are caused by the hyperplasia of adipocytes due to excessive differentiation from bone marrow mesenchymal stem cells into adipocytes.

The agent suppressing differentiation into adipocytes according to the present invention has the effect of suppressing adipocyte formation, the effect of suppressing fat accumulation, the effect of preventing obesity, the effect of suppressing cellulite formation, the effect of suppressing hyperlipemia, and the like; thus, it can be advantageously used as a pharmaceutical agent having these effects, as an agent for preventing or ameliorating symptoms in pathological conditions involving adipocyte hyperplasia, or further, as a pharmacological composition component to be added and blended into a food to make a functional food having the effect of preventing/ameliorating pathological conditions involving adipocyte hyperplasia.

The therapeutic effective amount of the agent suppressing differentiation into adipocytes according to the present invention is not particularly limited, provided that it is an amount capable of providing the effect of suppressing adipocyte formation to a subject in need thereof; for example, the effect of preventing/ameliorating pathological conditions due to adipocyte hyperplasia is provided by ingesting the water extract of Sargassum horneri (dry weight) in the range of 0.1 to 1,000 mg/kg body weight, preferably 1 to 100 mg/kg body weight, per day or by ingesting a dried product of Sargassum horneri in the range of 1 mg to 5 g/kg body weight, preferably 10 to 1,000 mg/kg body weight, per day. However, the amount to be ingested may be properly adjusted depending on symptoms, sex, age, and the like; examples of the therapeutic effective amount can include an amount for treating pathological conditions involving the adipocyte hyperplasia and, for convenience, may include an effective amount for preventing pathological conditions involving the adipocyte hyperplasia.

Examples of the form of administering the agent suppressing differentiation into adipocytes according to the present invention include forms of oral administration and forms of injecting dosage forms such solutions, emulsions and suspensions. The agent suppressing differentiation into adipocytes according to the present invention can be used as a pharmaceutical agent, a supplement, or a functional food useful for the treatment or prevention of a disease involving adipocyte hyperplasia.

When the agent suppressing differentiation into adipocytes according to the present invention is used as a pharmaceutical agent such as an agent suppressing adipocyte formation, various formulation ingredients can be added such as pharmaceutically acceptable common carrier, binder, stabilizer, excipient, diluent, pH buffer, disintegrator, solubilizer, solubilization agent, and isotonic agent to prepare an anti-obesity agent, an agent preventing the accumulation of fat such as visceral fat and subcutaneous fat, an anti-cellulite formation agent, a hypolipidemic agent, or the like; the agent can be used in combination with other therapeutic agents for other pathological conditions involving adipocyte hyperplasia, such as obesity and hyperlipemia.

The agent suppressing differentiation into adipocytes according to the present invention can be used for the production of a functional food or a food material for preventing/ameliorating the pathological condition of adipocyte hyperplasia. The agent suppressing differentiation can be added/blended during or after the production of the functional food or the food material.

Specific examples of the food can include various beverages such as yogurt, drink yogurt, juice, cow milk, soymilk, alcoholic drinks, coffee, black tea, green tea, oolong tea, and sports beverages; bakery such as pudding, cookie, bread, cake, jelly, baked goods (e.g., Japanese cracker), Japanese confectionery (e.g., sweet jelly made from bean jam), frozen dessert, and chewing gum; noodles such as wheat noodle and buckwheat noodle; fish meat paste products such as boiled fish paste, ham, and fish meat sausage; seasonings such as miso, soy sauce, dressing, mayonnaise, and sweetener; milk products such as cheese and butter; various delicatessens such as tofu, konjac, food boiled in soy sauce, gyoza (meat dumpling), croquette, and salad; and various beverages such as yogurt, drink yogurt, juice, cow milk, soymilk, alcoholic drinks, coffee, black tea, green tea, oolong tea, and sports beverages.

The agent suppressing differentiation into adipocytes according to the present invention can be used for the production of a feedstuff for preventing/ameliorating the pathological condition of adipocyte hyperplasia. The feedstuff for prevention/amelioration can be prepared by blending Sargassum horneri or a processed product thereof in a feedstuff for mice, rats, birds, pigs, sheep, cattle, cats, dogs, primates, or the like, more specifically, a feedstuff for livestock such as pigs, sheep and cattle, a feedstuff for poultry such as chickens, a diet feedstuff for cats, dogs, or the like, or an experimental feedstuff for mice, rats, or the like; feedstuff in which Sargassum horneri or a processed product thereof is blended as an active ingredient is useful for the prevention/amelioration of the pathological condition of adipocyte hyperplasia in livestock, poultry, pets, and experimental animals.

The present invention will be more specifically described below with reference to Examples. However, these Examples are not intended to limit the technical scope of the present invention.

### EXAMPLES

### Example 1

### Preparation of Water Extract of Sargassum Horneri

The marine algae Sargassum horneri was collected from coasts in Shimoda-city Shizuoka Prefecture Japan and Miyako-city Iwate Prefecture Japan. Sargassum horneri washed with water was homogenized (ground) in purified distilled water and then centrifuged at 5,500 g for 10 minutes, and the supernatant was freeze-dried in the form of a water-soluble extract of Sargassum horneri. The freeze-dried water-soluble extract of Sargassum horneri was dissolved in purified distilled water, and a fraction having a molecular weight of 3,000 or less was separated by a membrane fractionation method to prepare a water extract of Sargassum horneri. The water extract of Sargassum horneri was freeze-dried, and used by dissolution in purified distilled water when used for an experiment.

### (Preparation of Bone Marrow Mesenchymal Stem Cell)

Mouse bone marrow mesenchymal stem cells were collected as follows. Mice (CD1-Elite, wild type, 2-month old female) were purchased from Charles River Laboratories, Inc. (USA) and bred in an animal breeding room free of pathogenic bacteria. Thigh bones and neck bones obtained from the mice were subjected to the aseptic removal of muscle tissue in Dulbecco's Eagle culture medium (from Invitrogen Corporation (Carlsbad, Calif., USA)), and bone marrow cells were aseptically separated by subjecting the respective diaphyseal regions to desk-top centrifugation (at 10,000 rpm for 1 minute) and suspended in Dulbecco's Eagle culture medium to prepare a bone marrow cell suspension. The bone marrow cell suspension was further centrifuged at 1,200 rpm for 5 minutes and then washed. In addition, the resultant was subjected to erythrocyte removal treatment to provide mouse bone marrow mesenchymal stem cells.

[Study of Effect of Water Extract of Sargassum Horneri Suppressing Differentiation from Bone Marrow Mesenchymal Stem Cell into Adipocyte]

It was studied whether the water extract of Sargassum horneri suppressed differentiation from mouse bone marrow mesenchymal stem cells into adipocytes or not.

(Culture from Bone Marrow Mesenchymal Stem Cell into Preadipocyte—1)

To culture the mouse bone marrow mesenchymal stem cells (1×10⁶ cells/culture well/mL, 12-well plate), a culture medium for differentiation into preadipocytes was prepared by adding 1 μM/mL dexamethasone (from Sigma-Aldrich Co. LLC.) and 0.5 mM/mL 3-isobutyl-1-methylxanthine as cell differentiation-promoting factors to Dulbecco's Eagle culture medium containing 10% fetal bovine serum (from Hyclone, USA) and 1% penicillin-streptomycin (10,000 U/mL) (from Invitrogen Corporation (Carlsbad, Calif., USA)). The mouse bone marrow mesenchymal stem cells were cultured at 37° C. for 48 hours in the culture medium with 0 (control 1), 5, 10, 25, or 50 μg/mL of the water extract of Sargassum horneri. As control 2, the mouse bone marrow mesenchymal stem cells were similarly cultured using the culture medium not containing Sargassum horneri, and not containing dexamethasone or 3-isobutyl-1-methylxanthine.

(Culture from Preadipocyte into Adipocyte—1)

For the cells cultured for 48 hours, a culture medium for differentiation into adipocytes was prepared by adding 10 μg/mL insulin to Dulbecco's Eagle culture medium containing 10% fetal bovine serum and 1% penicillin-streptomycin. The cells were further cultured at 37° C. for 4 days in the culture medium with 0 (control 1), 5, 10, 25, or 50 μg/mL of the water extract of Sargassum horneri. For control 2, the preadipocytes were cultured using a culture medium not containing Sargassum horneri and not containing insulin.

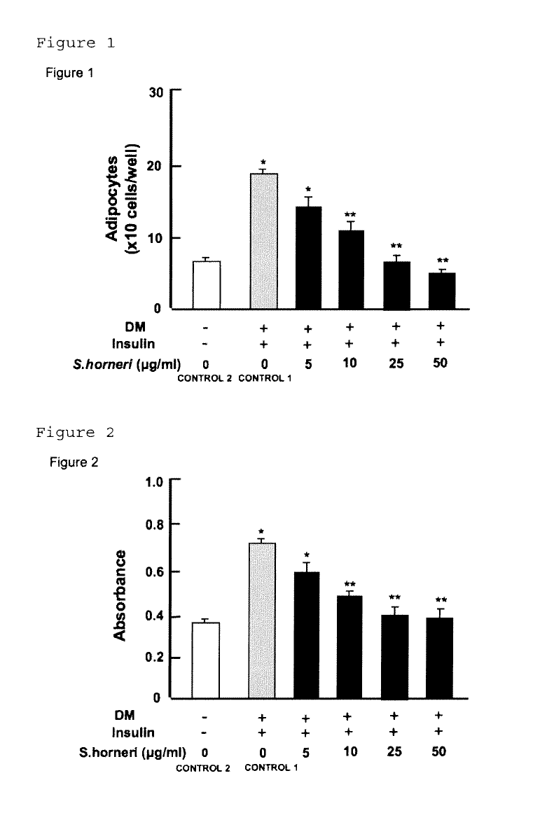

After removing the culture medium, cells were fixed with a 10% formalin solution, and Oil Red reagent was then added thereto to stain adipocytes to measure the number of adipocytes. The results are shown in FIG. 1. Thereafter, 0.2 mL of isobutyl alcohol was added to the Oil Red-stained cells, followed by dissolution by shaking for 10 minutes; the absorbance is shown which was measured at a wavelength of 490 nm using Spectra Count microplate photometer. Data are expressed in the average value and standard deviation of 8 samples for each group. * p<0.001 indicates a significant difference relative to the control 2 (white bar) for which no Sargassum horneri or cell differentiation-promoting factors were added, and ** p<0.001 indicates a significant difference relative to the control 1 (gray bar) for which the cell differentiation-promoting factors were added without adding Sargassum horneri. The results are shown in FIG. 2.

### (Result)

As is clear from FIG. 1, in the culture medium containing the water extract of Sargassum horneri, the number of formed adipocytes was confirmed to be significantly decreased in a manner dependent on the concentration thereof; particularly, the formation of adipocytes was significantly suppressed with the extract at a concentration of 10, 25, or 50 μg/mL culture medium, compared to the control 1. The number of adipocytes with the extract at a concentration of 25 or 50 μg/mL culture medium was comparable to or less than that with no cell differentiation-promoting factors. As is clear from FIG. 2, in the culture medium with the water extract of Sargassum horneri, the absorbance at 490 nm specifically absorbed by Oil Red was confirmed to be decreased in a manner dependent on the concentration thereof compared to the control 1, and to be significantly decreased particularly with the extract at a concentration of 10, 25, or 50 μg/mL culture medium, compared to the control 1. These results confirmed that the water extract of Sargassum horneri significantly suppressed differentiation from bone marrow mesenchymal stem cells into adipocytes and adipocyte formation.

### Example 2

### Study of Effect of Water Extract of Sargassum Horneri Suppressing Differentiation from Bone Marrow Mesenchymal Stem Cell into Preadipocyte

It was verified whether the water extract of Sargassum horneri prepared in Example 1 suppressed differentiation from bone marrow mesenchymal stem cells into preadipocytes or not.

(Culture from Bone Marrow Mesenchymal Stem Cell into Preadipocyte—2)

The mouse bone marrow mesenchymal stem cells prepared in Example 1 were cultured in each culture medium for 48 hours in the same procedure as that in “Culture from Bone Marrow Mesenchymal Stem Cell into Preadipocyte—1”. For control 2, a similar preparation was carried out.

(Culture from Preadipocyte into Adipocyte—2)

The cells cultured for 48 hours in each of the culture media were cultured for 4 days in the same procedure as that in “Culture from Preadipocyte into Adipocyte—1” except for replacement with a culture medium for differentiation into adipocytes, containing no water extract of Sargassum horneri.

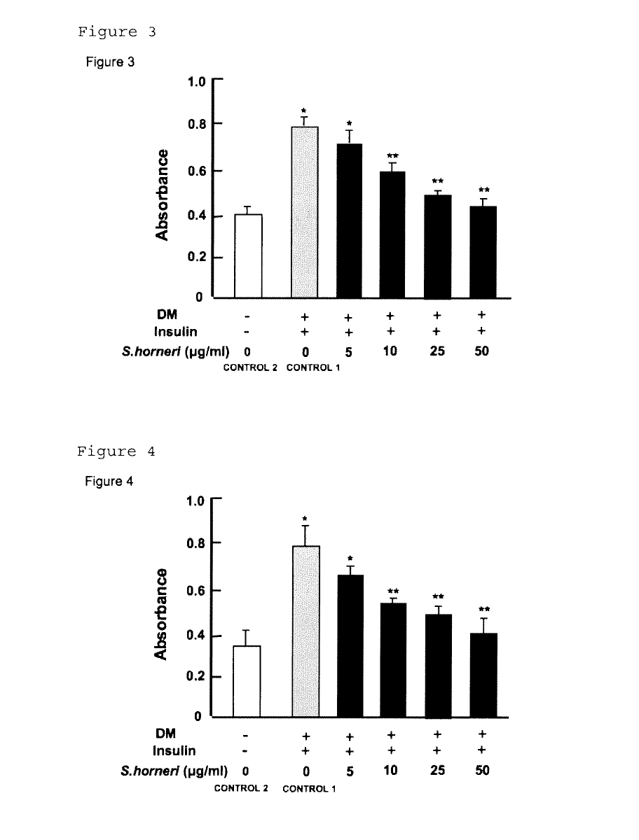

After removing the culture medium, cells were fixed with a 10% formalin solution, and Oil Red reagent was added thereto to stain adipocytes, and 0.2 mL of isobutyl alcohol was added to the Oil Red-stained cells, followed by dissolution by shaking for 10 minutes; the absorbance measured at a wavelength of 490 nm is shown. Data are expressed in the average value and standard deviation of 8 samples for each group. * p<0.001 indicates a significant difference relative to the control 2 (white bar) with no Sargassum horneri or cell differentiation-promoting factors, and ** p<0.001 indicates a significant difference relative to the control 1 (gray bar) with the cell differentiation-promoting factors but without Sargassum horneri. The results are shown in FIG. 3.

### (Result)

As is clear from FIG. 3, in the culture medium with the water extract of Sargassum horneri for differentiation into preadipocytes, the absorbance at 490 nm specifically absorbed by Oil Red was confirmed to be decreased in a manner dependent on the concentration thereof compared to the control 1 and to be significantly decreased particularly with the extract at a concentration of 10, 25, or 50 μg/mL culture medium, compared to the control 1. These results confirmed that the water extract of Sargassum horneri significantly suppressed differentiation from bone marrow mesenchymal stem cells into preadipocytes and adipocyte formation.

### Example 3

### Study of Effect of Water Extract of Sargassum horneri Suppressing Differentiation from Preadipocyte into Adipocyte

It was verified whether the water extract of Sargassum horneri prepared in Example 1 suppressed differentiation from preadipocytes into adipocytes or not.

(Culture from Bone Marrow Mesenchymal Stem Cell into Preadipocyte—3)

The mouse bone marrow mesenchymal stem cells prepared in Example 1 were cultured in each culture medium for 48 hours in the same procedure as that in “Culture from Bone Marrow Mesenchymal Stem Cell into Preadipocyte—1” except for culture in a culture medium for differentiation into preadipocytes, with no water extract of Sargassum horneri.

(Culture from Preadipocyte into Adipocyte—3)

The cells cultured for 48 hours were cultured for 4 days in the same procedure as that in “Culture from Preadipocyte into Adipocyte—1” of Example 1.

After removing the culture medium, cells were fixed with a 10% formalin solution, and Oil Red reagent was added thereto to stain adipocytes, and 0.2 mL of isobutyl alcohol was added to the Oil Red-stained cells, followed by dissolution by shaking for 10 minutes; the absorbance measured at a wavelength of 490 nm is shown. Data are expressed in the average value and standard deviation of 8 samples for each group. * p<0.001 indicates a significant difference relative to the control 2 (white bar) with no Sargassum horneri or cell differentiation-promoting factors, and ** p<0.001 indicates a significant difference relative to the control 1 (gray bar) with the cell differentiation-promoting factors but without Sargassum horneri. The results are shown in FIG. 4.

### (Result)

As is clear from FIG. 4, in the culture medium with the water extract of Sargassum horneri added in the differentiation stage into adipocytes, the absorbance at 490 nm specifically absorbed by Oil Red was confirmed to be significantly decreased in a manner dependent on the concentration thereof, particularly with the extract at a concentration of 10, 25, or 50 μg/mL culture medium, compared to the control. These results confirmed that the water extract of Sargassum horneri significantly suppressed differentiation from preadipocytes into adipocytes and adipocyte formation.

### DISCUSSION

The above results suggest the possibility that the Sargassum horneri extract acts on both the process of differentiation from bone marrow mesenchymal stem cells into preadipocytes and the process of differentiation from preadipocytes into adipocytes and exerts the effect of suppressing adipocyte formation, the effect of suppressing fat accumulation, the effect of preventing obesity, the effect of suppressing cellulite, and the effect of suppressing fat synthesis.

### INDUSTRIAL APPLICABILITY

The present invention can prevent or treat the pathological condition of adipocyte hyperplasia because it can use the marine algae Sargassum horneri or a processed product thereof, particularly a Sargassum horneri extract having a molecular weight of 3,000 or less, as an active ingredient to suppress differentiation from bone marrow mesenchymal stem cells into preadipocytes and further into adipocytes. It is highly safe because of being a preparation from Sargassum horneri as an algae having a long history as food, and can be expected not only to be conducive to a healthy life in individual's old age but also to contribute to cut in health care spending in an aging society because of being ingestible on a daily basis from one's early life for preventive purposes.

1. A method for suppressing adipocyte formation in a subject in need thereof, comprising administering a therapeutic effective amount of an agent suppressing differentiation into adipocyte to the subject, wherein the agent suppressing differentiation is Sargassum horneri or a processed product thereof.

2. The method for suppressing adipocyte formation according to claim 1, wherein the processed product of Sargassum horneri is an extract of Sargassum horneri.

3. The method for suppressing adipocyte formation according to claim 2, wherein the extract of Sargassum horneri is a water extract of Sargassum horneri.

4. The method for suppressing adipocyte formation according to claim 3, wherein the water extract of Sargassum horneri is a water extract of Sargassum horneri having a molecular weight of 3,000 or less.

5. The method for suppressing adipocyte formation according to claim 1, wherein the agent suppressing differentiation into adipocyte is an agent suppressing differentiation from bone marrow cell into preadipocyte.

6. The method for suppressing adipocyte formation according to claim 1, wherein the agent suppressing differentiation into adipocyte is an agent suppressing differentiation from preadipocyte into adipocyte.

7. The method for suppressing adipocyte formation according to claim 1, wherein the subject is one or more selected from mouse, rat, bird, pig, sheep, cattle, cat, dog, primate, and human.
